(19) United States
(12) Patent Application Publication
(10) Pub. No.: US 2008/0227763 A1
(43) Pub. Date: Sep. 18, 2008

(54) Topical hormonal composition with systemic action

(75) Inventors: Michel Lanquetin, La Trinite (FR); Jacques Paris, Nice (FR); Jean-Louis Thomas, Charenton Le Pont (FR)

Correspondence Address: Charles A. Muserlian;c/o Hedman and Costigan, 1185 Avenue of the Americas, New York, NY, 10036, US

(73) Assignee: LABORATOIRE THERAMEX

(21) Appl. No.: 12/077,665

(22) Filed: Mar. 20, 2008

Related U.S. Application Data
(62) Division of application No. 09/646,763, filed on Oct. 24, 2000.

(30) Foreign Application Priority Data
Mar. 23, 1998 (FR) 98/03533

(51) Int. Cl. A61K 31/57

(52) U.S. Cl. 514/177

(57) ABSTRACT

The present invention relates to the area of chemotherapy and more especially to the development of new galenic forms for application to the skin.

It relates more particularly to a topical hormonal composition with systemic action for the correction of progesterone deficiency in premenopausal women and for hormone replacement in menopausal women, characterized in that it contains, as the active principle, a progestogen derived from 19-nor progesterone, a vehicle permitting systemic passage of the said active principle chosen from the group comprising a solubilizing agent, an absorption promoter, a film-forming agent, a gelling agent or their mixtures, combined with or mixed with suitable excipients for production of a pharmaceutical form as a gel and/or a film.

The present invention relates to the area of therapeutic chemistry and especially to the development of new galenic forms for application on the skin.

The present invention relates more particularly to galenic preparations whose active principle is a synthetic progestogen, intended to be applied to the skin in order to achieve a systemic hormonal effect in women before and after the menopause.

Thus, the invention relates to a topical hormonal composition with systemic action.

In particular, French Patent 2.271.833 describes hormonal compositions for the correction of progestogen deficiencies in premenopausal or menopausal women, intended for oral administration.

However, the oral route is not without certain drawbacks for natural progesterone, as well as for synthetic progestogens. On the one hand, it requires the administration of quite large doses in order to make up for degradation of the active principle during passage through the intestine and in the liver (called the “first passage” effect). On the other hand, it does not give constant plasma levels over time since oral administration is followed by a plasma peak during which the blood concentrations are raised temporarily.

Natural progesterone is sometimes administered percutaneously. This route only produces local effects, and does not permit remote impregnation of the target tissues, notably the uterine mucosa. This is because there is rapid degradation of the hormone by enzymes in the subcutaneous tissue, making it impossible to reach sufficiently high plasma levels to produce a systemic hormonal action.

Many synthetic progestogens have the same drawback and cannot be used percutaneously to achieve a systemic effect. The only exception is norethisterone acetate administered in patches.

The skin's function as a protective barrier against external aggressive agents makes it rather impermeable to numerous substances and only allows medicinal molecules to penetrate under certain conditions: size and nature of the molecule, solubility, stability, nature of the vehicle containing the molecule, etc.

Thus, the release of an active principle from a vehicle and its penetration through the skin and as far as the blood or lymphatic circulation depend on numerous physicochemical and/or physiological parameters.

In the present invention, the very nature of the active principle (synthetic progestogen) presents the main obstacle to penetration through the skin: the main problem that arises is poor diffusion through the epidermis on account of its lipophilic character. The choice of vehicle used in the compositions will therefore have a considerable influence on percutaneous penetration and on the therapeutic activity of the molecule.

Thus, the topical compositions according to the invention permit a systemic effect by optimization of percutaneous passage of a synthetic progestogen derived from 19-nor progesterone.

The topical compositions according to the invention contain, as active principle, a synthetic progestogen derived from 19-nor progesterone and excipients that ensure optimum passage of the active principle through the skin.

The present invention relates more specifically to a topical hormonal composition with systemic effect for correcting progesterone deficiencies in premenopausal women and for hormone replacement in menopausal women, characterized in that it comprises:
as active principle, a progestogen derived from 19-nor progesterone,
a vehicle permitting systemic passage of the said active principle chosen from the group that includes a solubilizing agent, an absorption promoter, a film-forming agent, a gelling agent or their mixtures,
   combined with or mixed with suitable excipients for production of a pharmaceutical form as a gel and/or a film.

The compositions according to the invention can therefore be in the form of a gel, a film-forming gel or a film-forming solution.

The progestogen derived from 19-nor progesterone used in the present invention is nomegestrol and/or one of its esters or ethers.

An example of nomegestrol ether is tetrahydropyranic ether of nomegestrol.

An example of nomegestrol ester is nomegestrol acetate, which is a synthetic progestogen that is active in oral administration, whose action comprises correction of gynaecological disorders caused by deficiency of luteinizing hormones.

Administered by means of compositions according to the invention, nomegestrol acetate is able to pass through the skin and enter the blood circulation to give plasma levels that can be detected by the methods used for assaying in biological media. The plasma concentrations observed are maintained at a plateau after cutaneous application because of the reservoir effect of the skin.

The plasma levels of nomegestrol acetate obtained with the compositions according to the invention are able to create a hormonal effect on tissues located far from the site of application, and especially on the endometrium.

Repeated administration of nomegestrol acetate produces a therapeutic action when it is given to premenopausal women suffering from symptoms connected with progesterone deficiency or to menopausal women undergoing oestrogen replacement therapy.

According to the invention, nomegestrol or one of its esters or ethers is present in a quantity varying from 0.05 to 1 wt. % of the total composition. Preferably, nomegestrol or one of its esters or ethers is present in an amount varying from 0.1 to 0.8 wt. % of the total composition. The topical compositions with systemic effect that are preferred according to the invention are those containing a quantity of nomegestrol or of one of its esters or ethers of 0.4 wt. % of the total composition.

The solubilizing agents and the absorption promoters have different modes of action but they both favour penetration of the active principle through the skin.

The solubilizing agents improve the solubility of the active principle and alter its affinity for the skin by acting upon the thermodynamic activity of the active molecule.

The absorption promoters lower the resistance to diffusion by modifying the structure of the cutaneous barrier.

However, there is no direct relation between improvement of solubility of the active principle in the vehicle and increased passage through the skin. In fact, the use of agents that improve the solubility of the active principle also increases its affinity for the vehicle and therefore generally lowers its diffusion through the skin.

Thus, for the solubility of the active principle in a vehicle to be total, there must be a certain affinity for the latter; however, it must not be too great, so that division of the active principle is oriented towards its diffusion through the skin.

According to the present invention, examples of suitable solubilizing agents are water, alcohols, propyleneglycol, polyethylene glycol, polyethylene 20 sorbitan mono-oleate (marketed for example under the name Polysorbate 80 DF), a C₈/C₁₀ polyoxyethylene glycosyl glyceride (marketed for example under the trade-name Labrasol®) or their mixtures.

The solubilizing agent used is generally a mixture of solvents or of the aforementioned solubilizing agents, which, by synergistic action, is more effective than one of them used alone. The solubilizing agent is preferably chosen from the group comprising water, alcohols, propyleneglycol, a C₈/C₁₀ polyoxyethylene glycosyl glyceride or their mixtures. Thus, it will be possible to use, as solubilizing agent, a binary mixture of 95° ethanol and water, in which the percentage of 95° ethanol varies from 30 to 50%, and especially a binary mixture of 95° ethanol and water in which the percentage of 95° ethanol is 45%.

However, particularly preferred examples of solubilizing agents suitable for the topical composition with systemic effect according to the invention are:
a ternary mixture 95° ethanol/water/propyleneglycol, in which the percentage of 95° ethanol varies from 30 to 50%, that of water from 30 to 60%, and that of propyleneglycol from 2 to 20%; preferably, the percentage of 95° ethanol is 45%, that of water is 45%, and that of propyleneglycol is 8%,
a quaternary mixture 95 ethanol/water/Labrasol®/propyleneglycol, in which the percentage of 95 ethanol varies from 30 to 50%, that of water from 30 to 60%, that of Labrasol® from 3 to 7% and that of propyleneglycol from 2 to 20%;
   preferably, the percentage of 95° ethanol is 45%, that of water is 33.5%, that of Labrasol® is 5% and that of propyleneglycol is 15%;

Of the substances regarded as absorption promoters, or “enhancers”, the most used are derivatives of glycol, sulphoxides, surfactants, fatty acids and terpene derivatives.

As examples of absorption promoters, we may mention oleic acid, oleic alcohol, a triglyceride of decanoic and octanoic acids (for example, as marketed under the trade-name Miglyol 812®), isopropyl myristate, propyleneglycol dipelargonate, 2n-nonyl-1.3-dioxolane, octyl dodecyl myristate, isopropylidene glycerol (for example as marketed under the trade-name Solketal), α-tocopheryl propyleneglycol 1000 succinate (for example as marketed under the trade-name Vitamin E TPGS), monoethyl ether of diethyleneglycol (for example as marketed under the trade-name Transcutol®).

The absorption promoter that is more particularly suitable in the present invention is chosen from the group comprising isopropylidene glycerol, α-tocopheryl propyleneglycol 1000 succinate and monoethyl ether of diethyleneglycol.

However, the preferred absorption promoter is isopropylideneglycerol.

The forms envisaged for ensuring penetration of the active principle through the skin will be either gels, or occlusive gelled preparations.

The choice of gelling agents and film-forming agents is also important in the compositions according to the invention.

The gelling agents are substances which thicken and alter the viscosity of a liquid vehicle thus constituting a three-dimensional colloidal network, the gel.

There are various kinds of gelling agents: natural gelling agents (mineral, vegetable, animal), synthetic agents and semi-synthetic agents.

Examples of natural gelling agents are guar gum, extracts from algae (alginates, carrageenans, agar), polysaccharides (xanthan gum, gum arabic, tragacanth), starches, pectins, etc.

Examples of synthetic or semi-synthetic gelling agents are cellulose derivatives, especially those obtained by esterification or etherification of cellulose, and acrylic derivatives. The category of acrylic derivatives includes carbomers, polycarbophils, and acrylates.

In the present invention, the gelling agent is chosen from the group comprising cellulose derivatives and acrylic derivatives.

The cellulose derivatives include:
methylcelluloses (Methocel, Metolose),
ethylcelluloses (Ethocel, Aquacoat®)
hydroxypropylmethylcelluloses (Kenal Methocel, Hypromelose),
hydroxyethylcelluloses (Cellosize, Natrosol),
hydroxypropylcelluloses (Klucel),
carboxymethylcelluloses in the sodium or calcium form (Akucell, Nymcel Tylose CB),

The choice of a polymer is made from the Metolose range, from the company Shin Etsu. For each of these types, there are different degrees (or grades) depending on the substituents and the degree of substitution, which give different viscosities to the polymer solutions. There is a classification of the celluloses according to their adhesive potential. The choice of grade is important because the adhesive power of the cellulose derivative varies in relation to the latter. According to the present invention, a particularly suitable cellulose derivative is hydroxypropylmethylcellulose, and especially hydroxypropylmethylcellulose of grade 60 SH 4000. In fact, grade 60 SH has the most suitable properties: good solubility in organic solvents and high resistance to electrolytes. It also makes it possible to obtain transparent gels. Among the acrylic derivatives, the carbomers are particularly suitable according to the present invention, and especially those marketed under the trade-names Carbopol® or Synthalen®.

The carbomers give formulations that are stable over time, and endow the formulation with reproducible rheological properties on account of their synthetic nature.

The existence of different degrees or grades results from the difference in molecular weight, degree of crosslinking, nature of the molecular arrangements and the polymerization solvent.

Thus, among the various grades of carbomers, we may mention those marketed by the Goodrich company under the trade-names Carbopol 974 P®, Carbopol 980®, Carbopol 1382® and Carbopol 2020®, or similar products such as the Synthalens from 3 V France, as they are (Synthalen K, L, M) or preneutralized, for example Synthalen PNC®.

However, according to the present invention, the carbomers marketed under the trade-names Carbopol 980®, Carbopol 1382® and Synthalen K® are particularly suitable and offer considerable advantages, as they are fluidized in contact with the electrolytes of the skin and thus prevent deposition of polymer, which could hamper passage of the active principle.

The film-forming agents used are those that are employed for producing solutions for enrobing and coating, as most of them are obtained from the biomedical or food industry and are suitable for human application.

These film-forming agents can be placed in different groups according to their solubility.

Whatever the film-forming agent, the quality of the film-forming gel obtained or of the film-forming solution obtained depends on the percentage of film-forming agent, the type of solvent, the presence and nature of the plasticizer.

According to the present invention, the film-forming agent is chosen from the group comprising cellulose derivatives, methacrylic derivatives and polyvinylpyrrolidone derivatives.

Among the cellulose derivatives, we may mention:
hydroxypropylmethylcellulose acetate succinate, and especially that marketed by the Seppic company under the trade-name Aqoat AS-LF®,
an aqueous dispersion of cellulose acetophthalate containing 70% water, 23% cellulose acetophthalate and 7% poloxamer, and especially that marketed by the Seppic company under the trade-name Aquacoat CPD®,
an aqueous dispersion of ethylcellulose, cetyl alcohol and sodium lauryl sulphate, and especially that marketed by the Seppic company under the trade-name Aquacoat ECD 30®,
ethylcellulose.

Among the methacrylic derivatives, we may mention:
an aqueous dispersion of an anionic copolymer of methacrylic acid and ethyl acrylate (type C), especially that containing 30% of dry copolymer, 0.7% of sodium lauryl sulphate and 2.3% of Polysorbate 80 NF, and marketed under the trade-name Eudragit L30 D550® (Rohm & Haas),
a copolymer of acrylic acid and methacrylic ester (type A), especially that marketed under the trade-name Eudragit RL 100® (Rohm & Haas).

Among the derivatives of polyvinylpyrrolidone, we may mention:
a povidone, of formula (C₆H₉NO)ₙ whose molecular weight is of the order of 360 000, marketed under the trade-name Kollidon 90®
polyvinylpyrrolidone/vinylacetate 64 copolymer, of formula (C₆H₉NO)ₙ×(C₄H₆O₂)ₘ whose molecular weight is: (111.1)ₙ×(86.1)ₘ.
homopolymers of polyvinyl alcohol

In the present invention, the particularly suitable cellulose derivative is hydroxypropylmethylcellulose acetate succinate, the particularly suitable methacrylic derivative is an aqueous dispersion of an anionic copolymer of methacrylic acid and ethyl acrylate, and the particularly suitable derivative of polyvinylpyrrolidone is a povidone.

The topical hormonal compositions with systemic effect according to the invention can additionally contain other excipients that are complexing agents, neutralizing agents such as disodium edetate (EDTA), triethanolamine (TEA) and/or plasticizers such as diethyl phthalate and triacetin.

A particularly suitable topical hormonal composition according to the invention is a composition in the form of gel or film-forming gel, with a content of nomegestrol or nomegestrol acetate of 0.4 wt. % of the total composition, a pH between 6 and 7, and a viscosity between 1000 and 2000 mPas.

The method of preparation of the compositions with systemic effect according to the invention varies depending on the particular nature of the compositions that are to be produced, namely a gel, a film-forming gel or a film-forming solution.

### METHOD OF PREPARATION OF THE GELS

Similarly, during the preparation of compositions in the form of gel, the manner of preparation will not be exactly the same, depending on the type of gelling agent used. Thus, in the preparation of gels, with regard to the gelling agent a distinction is made between synthetic acrylic derivatives and cellulose derivatives.

Preparation from Acrylic Derivatives

The important steps in the preparation of a gel are dispersion of the gelling agent in the solubilizing agent (this dispersion will largely determine the quality of the preparation obtained), stirring, hydration, swelling and finally gelling.

### Dispersion and Stirring: Wetting

The acrylic derivative is suspended with stirring in the solvent (solubilizing agent). Stirring must be moderate, otherwise the acrylic polymer is degraded by shearing and loses its efficacy.

### Hydration and Swelling of the Polymers

To prevent the formation of partially hydrated lumps, it is recommended to incorporate the polymers by sieving, to facilitate wetting and hydration of the powder and permit them to form a network. This step is promoted by wetting the powder beforehand, in the most polar solvent in the case of a solvent system.

### Gelling: Neutralization of the Dispersion Obtained

The pH of such a suspension is close to 3 (the pH is a function of the concentration of polymer, and therefore of carboxyl groups). Mineral bases such as sodium hydroxide, potassium hydroxide or ammonium hydroxide are used when the solvents in the formulation are aqueous, and organic bases such as amines (triethanolamine, tromethamine or TRIS etc.) when they are nonpolar or only slightly polar. Addition of these agents causes spontaneous thickening through formation of water-soluble salts of polymer resins.

An example of preparation of a gel whose gelling agent is an acrylic derivative is characterized in that:
nomegestrol acetate and EDTA are dissolved in the solvent system water/95° ethanol/propyleneglycol with stirring at 300 rev/min (# 30 min);
the acrylic polymer is dispersed in small portions in the solution of active principle with stirring at 100 rev/min;
the acrylic polymer is left to swell for 2 hours with stirring at 200 rev/min;
the dispersion is neutralized with triethanolamine dissolved in a portion of water taken from the quantity to be incorporated in the formulation; stirring is slowed to 100 rev/min during neutralization to avoid incorporation of air bubbles;
it is stirred for 30 min at 150 rev/min to homogenize the gel obtained.
   Preparation from Cellulose Derivatives

Gels formulated on the basis of cellulose derivatives do not need to be neutralized, but it will sometimes be necessary to adjust their pH by means of organic amines or inorganic hydroxides, depending on the type of solvent in the formulation.

The viscosity obtained depends on the type and quantity of cellulose derivative used.

An example of preparation of a gel whose gelling agent is a cellulose derivative is characterized in that:
nomegestrol acetate and EDTA are dissolved in the solvent system water/95° ethanol/propyleneglycol with stirring at 300 rev/min (# 30 min);
the cellulose polymer is dispersed in small portions in the solution of active principle with stirring at 100 rev/min;
the cellulose polymer is left to swell for 1 hour with stirring at 250 rev/min;
the pH is adjusted, if necessary, with triethanolamine dissolved in water with stirring at 100 rev/min;
it is stirred for 30 min at 150 rev/min to homogenize the gel obtained.

### FILM-FORMING GELS (or “FILMING GELS”) AND FILM-FORMING SOLUTIONS (or “FILMING SOLUTIONS”)

These forms are envisaged because, when they are applied to the skin, on drying they form a kind of occlusive film, which is sufficient to increase the hydration of the skin and create new sites for passage, thus improving the diffusion of the active principle that they contain. However, the form obtained must penetrate or dry quickly, leaving a pleasant, non-sticky feel. The film-forming agents used in the present invention are generally those used for production of enrobing solutions for tablets.

### METHOD OF PREPARATION OF FILM-FORMING SOLUTIONS

As with gels, in the preparation of compositions in the form of a film-forming solution, the manner of preparation will vary depending on the nature of the film-forming agent used.

Preparation from Solid Film-Forming Agent:

The steps in preparation are:
Dissolving of a plasticizer and of the active principle in the solvent mixture: the mixture containing the plasticizer and the active principle must be stirred for a sufficient time to obtain a solution.
Dispersion and solution of the film-forming agent:

Dispersion must be carried out in small portions, with vigorous stirring. Stirring is continued until the film-forming agent has dissolved completely. Neutralization of the film-forming solution is carried out, if necessary, at the end of production, with slower stirring.

First, two solutions are prepared separately:
an aqueous solution containing a dissolved plasticizer, in which the film-forming agent is dissolved completely with vigorous stirring;
an alcoholic solution containing the other excipients of the formulation and in which the active principle is dissolved; the gelling agent is dispersed in it and left to swell.

Then the alcoholic solution is mixed into the aqueous solution and the solution is gelled with triethanolamine.

An example of preparation of a film-forming gel is characterized in that:
the plasticizer is dissolved in water; stirring is carried out for 30 min at 250 rev/min;
the film-forming agent is dispersed in it and stirred at 250 rev/min until it has dissolved completely (in the case of a solid film-forming agent) or until the dispersion is homogeneous; stirring is continued for 1 hour;
separately, the EDTA and nomegestrol acetate are dissolved in the mixture of propyleneglycol and ethanol; the chosen gelling agent is dispersed and is left to swell for 2 hours, stirring at 150 rev/min;
the alcoholic solution is mixed with the aqueous solution, and stirred for 1 hour at 150 rev/min;
neutralization is carried out with triethanolamine dissolved in water, lowering the speed of stirring to 100 rev/min; the gel obtained is homogenized by stirring for 30 min.

### METHOD OF EVALUATING PASSAGE OF THE ACTIVE PRINCIPLE THROUGH THE SKIN

The efficacy of the topical composition according to the invention is evaluated by demonstrating that the active principle it contains diffuses through the skin and is absorbed into the microcirculation in sufficient quantity to achieve the desired therapeutic effect.

In the present invention, passage of nomegestrol acetate through the skin is evaluated by measuring the radioactivity, using a molecule labelled with carbon 14. The method of evaluation of passage of the active principle using radiolabelled products makes it possible to detect low levels of the active principle, which represents a considerable advantage, bearing in mind the small quantities that diffuse through the skin.

An example of preparation of a film-forming solution whose film-forming agent is solid is characterized in that:
the quantities of ethanol, water and propyleneglycol required for the formulation are stirred at 250 rev/min for 10 min;
EDTA and nomegestrol acetate are dissolved in the mixture obtained;
the plasticizer is added and stirring is continued for 30 min at 250 rev/min;
the film-forming agent is dispersed in small portions, stirring at the same speed, until it has dissolved completely; stirring is then continued for 1 hour;
the pH is adjusted by means of a solution of triethanolamine dissolved in a small amount of water, taken from the quantity of water to be incorporated in the formulation, reducing stirring to 100 rev/min; the solution obtained is homogenized for 30 min.
   Preparation from a Film-Forming Agent in Aqueous Dispersion

The steps in preparation are:
Solution and plasticizing of the film-forming agent
Incorporation of the mixture containing the active principle and the other excipients in small portions, with vigorous stirring

Neutralization is carried out at the end of production, with slower stirring.

An example of preparation of a film-forming solution whose film-forming agent is an aqueous dispersion is characterized in that:
the water and a plasticizer are mixed at 250 rev/min; stirring for 30 min;
the dispersion of film-forming agent is added in small portions, stirring at 250 rev/min, until a homogeneous solution of the dispersion is obtained; stirring is continued for 1 hour;
separately, EDTA and nomegestrol acetate are dissolved in the ethanol/propyleneglycol mixture; stirring is continued until they are dissolved completely;
the alcoholic solution of active principle is added in small portions to the aqueous solution, with stirring at 250 rev/min; the solution obtained is stirred for 1 hour to homogenize it;
the solution is neutralized with triethanolamine dissolved in water, with slower stirring; the solution obtained is homogenized for 30 min.

### METHOD OF PREPARATION OF FILM-FORMING GELS OR GELLED FILMS

The film-forming gels are obtained by gelling of film-forming solutions.

The skin used in the various tests for evaluating percutaneous passage of the active principle was obtained from abdominal plastic surgery on female subjects in the age range from 40 to 45 years. Excess adipose tissue is removed from the skin, which is then cleaned and stored in a freezer at −70° C.

The topical compositions according to the invention are intended to be applied mainly to the skin of the abdomen, arms, thighs, etc.

### EXPERIMENTAL SECTION

### Example I

FIG. 1 illustrates passage of the active principle nomegestrol acetate (NAc) through the skin as a function of different quantities of nomegestrol acetate in the compositions according to the invention.

The symbols ▪, ♦, and □ in FIG. 1 represent:

| | | |
|---|---|---|
| ▪ Gel 0.11% of NAc | ♦ Gel 0.4% of NAc | □ Gel 0.8% of NAc |

These compositions are in the form of gel and their formulations are given in Table 1 below:

TABLE 1

| FORMS | | | |
|---|---|---|---|
| FORMULATIONS (in %) | GELS | | |
| Nomegestrol Acetate | 0.40 | 0.80 | 0.11 |
| Propyleneglycol | 6.00 | 6.00 | 3.00 |
| Transcutol ® | 5.00 | 5.00 | — |
| Carbopol 1342 ® | 0.50 | 0.50 | — |
| Carbopol 940 ® | / | / | 0.75 |
| EDTA | 0.05 | 0.05 | 0.05 |
| Triethanolamine (TEA) | 0.30 | 0.30 | 0.30 |
| Demineralized water | 42.75 | 42.35 | 45.79 |
| Ethanol | 45.00 | 45.00 | 50.00 |
| pH (at 1%) | 6.7 | 6.5 | 6.7 |
| NOMEGESTROL ACETATE (mg/g) | 0.41 | 0.4 | 0.403 |

Passage of the active principle through the skin is evaluated by measuring the amount of active principle accumulated as a function of time. The amount of active principle accumulated represents the total diffusion of the active principle through the skin over a specified period (24 or 48 h). In this example, it is expressed in ng.

FIG. 1 clearly shows that the poorest diffusion results are obtained with the gel with 0.11% of NAc.

This gel at 0.11% was tested in preliminary clinical trials: cf. example IV. Thus, it was established that this gel, despite its poorer results, was still able to achieve a systemic passage effect.

### Example II

### Investigation of the Solubility of Nomegestrol Acetate (NAc)

### 1)-a) In a 95° Ethanol/Water Binary Mixture

The most effective system of solvent in aqueous-alcoholic mixture is determined.

TABLE 2
Solubility of nomegestrol acetate as a function
of the percentage of 95° ethanol

| | Solubility of nomegestrol |
|---|---|
| % 95° ethanol | acetate in mg/ml |
| 0 | 0.056 |
| 10 | 0.070 |
| 20 | 0.113 |
| 30 | 0.683 |
| 40 | 2.820 |
| 50 | 7.330 |
| 60 | 17.850 |
| 70 | 24.850 |
| 80 | 29.500 |
| 90 | 26.600 |
| 100 | 32.850 |

In aqueous-alcoholic mixture, solubility increases with the percentage of alcohol. The solubility profile shows that it is fairly low up to 40% alcohol, then increases sharply between 40 and 80%. Now, the percentage of alcohol permitted for forms for topical application is limited. Within these limits, the most effective solvent system for solubilizing nomegestrol acetate is between 40 and 60% of alcohol.

### b) In a Ternary Mixture 95° Ethanol/Water/Propyleneglycol

The effect of a ternary mixture of solvents, ethanol/water (45:55)/propyleneglycol on the solubility of nomegestrol acetate was determined.

We also examined the possibility of lowering the proportion of alcohol in the solvent, by means of this ternary mixture, while maintaining similar solubility; for this we choose the influence of propyleneglycol on solubility in ethanol/water systems (40:60 and 30:70).

TABLE 3
Solubility of nomegestrol acetate in various
systems containing propyleneglycol (PG)

| % Propylene- | Solubilities (mg/ml) | | |
|---|---|---|---|
| glycol | System I | System II | System III |
| 0 | 0.6 | 2.9 | 5.1 |
| 2 | 0.6 | 2.6 | 5.1 |
| 4 | 0.5 | 2.6 | 5.1 |
| 6 | 0.7 | 3.0 | 5.1 |
| 8 | 1.0 | 3.2 | 7.7 |
| 12 | 1.1 | 3.4 | 7.7 |
| 20 | 1.5 | 3.9 | 7.9 |

| | | |
|---|---|---|
| | | Ethanol 95°: 30% |
| System I: | {open oversize bracket} | |
| | | Demineralized water: 70% |
| | | Ethanol 95°: 40% |
| System II: | {open oversize bracket} | |
| | | Demineralized water: 60% |
| | | Ethanol 95°: 45% |
| System III: | {open oversize bracket} | |
| | | Demineralized water: 55% |

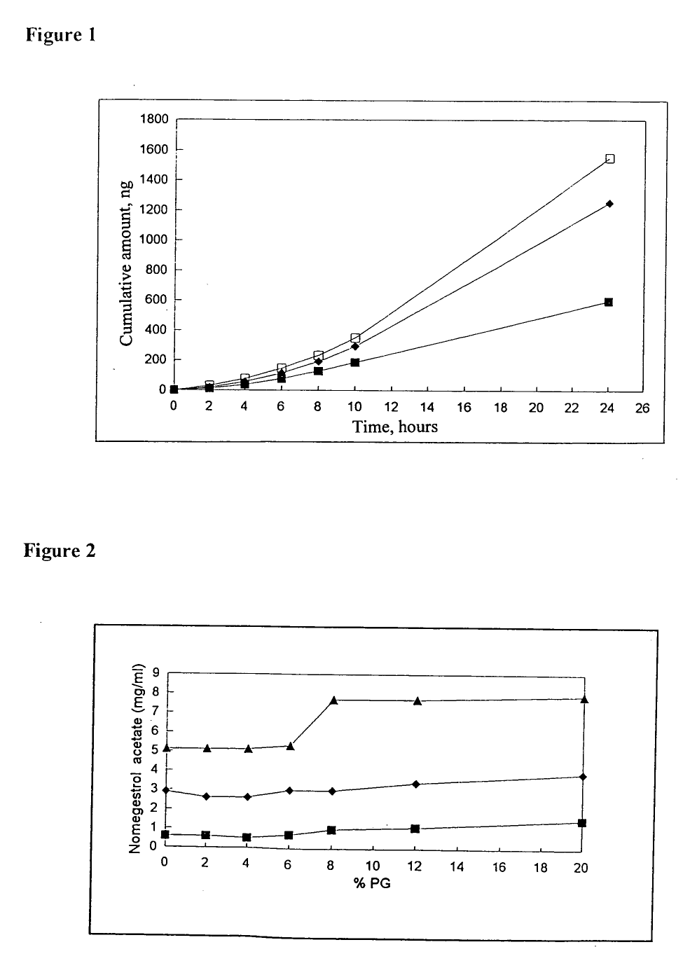

Table 3 is illustrated by FIG. 2.

The symbols ▪, ♦, and ▴ in FIG. 2 represent:

▪ Solubility in system I
♦ Solubility in system II
▴ Solubility in system III

In water/ethanol/propyleneglycol ternary mixture, the solubility of the active principle is improved with proportions of 8% of propyleneglycol for a mixture with 45% alcohol. It is for this system that we obtain the best solubility of the active principle. Propyleneglycol acts in synergy with the alcohol, on the solubility of nomegestrol acetate.

### c) In 95° Ethanol/Water/Labrasol/Propyleneglycol Mixture

TABLE 4
Solubility of nomegestrol acetate in
a system containing propyleneglycol

| | Solubility of l'nomegestrol |
|---|---|
| % Propyleneglycol | acetate (mg/ml) |
| 0 | 9.4 |
| 10 | 9.5 |
| 15 | 10.2 |

| | | |
|---|---|---|
| | | Ethanol 95°: 45% |
| System: | {open oversize bracket} | Water: 50% |
| | | Labrasol ®: 5% |

Using Labrasol® alone at 5% without propyleneglycol, the solubility increases in comparison with the results obtained with propyleneglycol. This solubility is increased further by combining propyleneglycol and Labrasol®.

### 2)-a) In a 95° Ethanol/Water/Propyleneglycol/Solketal Mixture

TABLE 5
Solubility of nomegestrol acetate in an aqueous-alcoholic
mixture containing propyleneglycol and/or Solketal

| | | Solubility of nomegestrol |
|---|---|---|
| % Propyleneglycol | % Solketal | acetate (mg/ml) |
| 0 | 3 | 6.7 |
| 0 | 8 | 8.6 |
| 8 | 0 | 7.7 |
| 8 | 3 | 10.7 |

The solubility of nomegestrol acetate in the aqueous-alcoholic solvent mixture in the presence of 8% of Solketal is greater than that obtained in the presence of 8% of propyleneglycol alone. Combination of the two substances propyleneglycol and Solketal greatly increases solubility in the aqueous-alcoholic solvent mixture, in the proportion of 8% of propyleneglycol/3% of Solketal.

### b) In a 95° Ethanol/Water/Propyleneglycol/Vitamin E TPGS Mixture

TABLE 6
Solubility of nomegestrol acetate in an aqueous-alcoholic
mixture containing propyleneglycol and/or vitamin E TPGS

| | | Solubility of nomegestrol |
|---|---|---|
| % Propyleneglycol | % Vitamin E TPGS | acetate (mg/ml) |
| 0 | 3 | 7.65 |
| 0 | 8 | 11.10 |
| 8 | 0 | 7.70 |
| 8 | 3 | 12.50 |

The solubility of nomegestrol acetate is improved in the presence of vitamin E TPGS alone, relative to propyleneglycol, for a same proportion of 8%. Incorporated at 3%, it already gives results equivalent to propyleneglycol used at 8%.

However, even better solubility is obtained when these two substances are combined in the proportion of 8% of propyleneglycol/3% of vitamin E TPGS.

### c) In a 95° Ethanol/Water/Propyleneglycol/Transcutol® Mixture

TABLE 7
Solubility of nomegestrol acetate in an aqueous-alcoholic mixture
containing propyleneglycol and/or Transcutol ®

| | | Solubility of nomegestrol |
|---|---|---|
| % Propyleneglycol | % Transcutol ® | acetate (mg/ml) |
| 8 | 0 | 7.70 |
| 8 | 3 | 7.95 |
| 0 | 8 | 10.70 |
| 3 | 8 | 10.60 |

The solubility of nomegestrol acetate is improved in the presence of 8% of Transcutol® alone, 20 relative to propyleneglycol at the same proportion. On combining these two substances, the same solubility is obtained using the proportions 8% Transcutol/3% propyleneglycol.

Reversing the proportions does not improve the solubility of the active principle in comparison with that obtained in propyleneglycol alone.

### Conclusion:

Aqueous-alcoholic mixtures containing:
8% of propyleneglycol and 3% of Solketal,
or 8% of propyleneglycol and 3% of vitamin E TPGS,
or 3% of propyleneglycol and 8% of Transcutol®,
   are particularly suitable for good solubility of the active principle.

### Example III

### 1/ Investigation of Formulations in the Form of Gels

Among the substances chosen for their qualities as absorption promoters, Solketal and vitamin E TPGS are particularly suitable as they also prove capable of improving the solubility of nomegestrol acetate aqueous-alcoholic mixture and propyleneglycol. The promoting action of the three promoters was investigated by incorporating them in formulations containing an aqueous-alcoholic gel at 45% alcohol and including 8% of propyleneglycol and 3% of the promoter. These formulations in the form of gels were tested for passage through the skin.

The gels used meet the requirements for pH, viscosity, concentration and appearance.

The four gels investigated were designated << G36-264, G36-276, G32-104 and G37-113 >> and their formulations are presented in Table 8 below:

TABLE 8

| | GELS | | | |
|---|---|---|---|---|
| | References | | | |
| FORMS | | | G32-104 | G37-113 |
| Formulations in % | G36-264 | G36-276 | reference | reference |
| Nomegestrol Acetate | 0.4 | 0.4 | 0.4 | 0.4 |
| Propyleneglycol | 8 | 8 | 8 | 8 |
| Transcutol ® | / | / | 3 | 3 |
| Solketal | 3 | / | / | / |
| Vit E TPGS | / | 3 | / | / |
| HPMC 60SH4000 | / | / | / | / |
| Carbopol 1382 ® | / | / | 0.5 | / |
| Carbopol 980 ® | 0.5 | 0.5 | / | 0.6 |
| EDTA disodium edetate | 0.05 | 0.05 | 0.05 | 0.05 |
| TEA | 0.4 | 0.3 | 0.3 | 0.25 |
| Kollidon 90 ® | / | / | / | / |
| Aqoat AS-LF ® | / | / | / | / |
| Eudragit L30D55 ® | / | / | / | / |
| Diethyl phthalate | / | / | / | / |
| Ethanol 95 | 45 | 45 | 45 | 45 |
| Demineralized water | 42.65 | 42.75 | 42.75 | 42.85 |
| pH solution at 1% | 6.9 | 6.92 | 6.6 | 6.37 |
| Viscosity mPa · s | 1150 | 1020 | 1400 | 1400 |
| NAc content (%) | 0.41 | 0.4 | 0.403 | 0.393 |

The main differences in the composition of these gels relate firstly to the choice of absorption promoter (“enhancer”) and secondly to the choice of gelling agent, which is either Carbopol 980® or Carbopol 1382®.

Passage of the active principle through the skin is evaluated by measuring:
the cumulative amount of active principle as a function of time (cf. example I),
the cumulative percentage of active principle as a function of time,
and the rate of diffusion of active principle as a function of time.

The cumulative percentage of active principle is the total percentage of diffusion of the active principle through the skin in a given time interval.

The rate of diffusion of the active principle is expressed in μg/cm²/h: it can be used for determining the kinetics of diffusion of the active principle over time.

The method used for evaluating passage of the active principle also makes it possible to determine the distribution of nomegestrol acetate in the various structures of the skin after diffusion.

Table 9 shows the cumulative percentage of nomegestrol acetate as a function of time, as well as (cf. last 3 lines) the sites of distribution of the active principle in the skin structures, i.e. the content of nomegestrol acetate in the various layers (epidermis+dermis) of the skin.

TABLE 9

| | cumulative % in μg | | | |
|---|---|---|---|---|
| | GELS | | | |
| | G37-113 | G32-104 | G36-276 | G36-264 |
| | 8% PG | 8% PG | 8% PG | 8% PG |
| | 3% Trans | 3% Trans | 3% Vit E | 3% Solketal |
| Time, h | 0.6% C980 | 0.5% C1382 | 0.5% C980 | 0.5% C980 |
| 0 | 0 | 0 | 0 | 0 |
| 2 | 0.042 | 0.058 | 0.105 | 0.183 |
| 4 | 0.088 | 0.135 | 0.193 | 0.371 |
| 6 | 0.137 | 0.227 | 0.275 | 0.554 |
| 8 | 0.19 | 0.329 | 0.347 | 0.732 |
| 10 | 0.239 | 0.402 | 0.405 | 0.894 |
| 24 | 0.575 | 1.117 | 0.667 | 1.926 |
| Epidermis | 10.05 | 14.82 | 8.15 | 16.82 |
| Dermis | 4.33 | 6.97 | 4.68 | 4.84 |
| Washing | 67.63 | 61.15 | 72.69 | 60.35 |

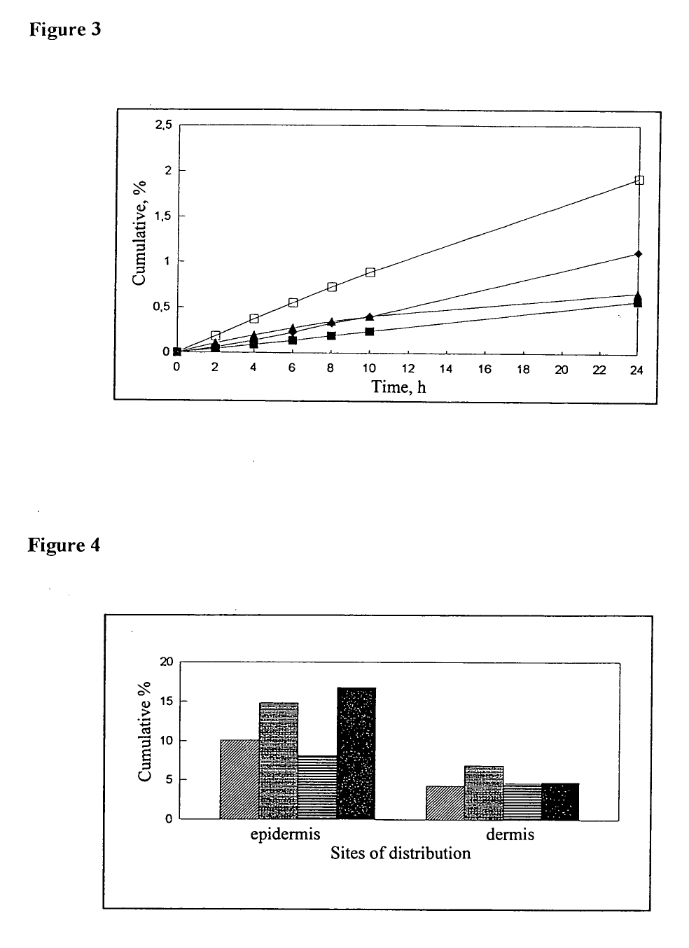

Table 9 is illustrated by FIGS. 3 and 4.

FIG. 3 shows the influence of enhancer (promoter) and of Carbopol® on passage of the systemic gel of nomegestrol acetate through the skin.

The following promoters were compared: Transcutol® (Tr), Solketal (Sol) and Vitamin E TPGS (Vit E).

The symbols ▪, ▴, ♦ and □ in FIG. 3 represent:

| | |
|---|---|
| ▪ G37-113 (3% Tr) | ♦ G32-104 (3% Tr) - Ref. |
| ▴ G36-276 (3% Vit E) | □ G36-264 (3% Sol) |

FIG. 4 shows the distribution of nomegestrol acetate in the structures of the skin.

The symbols [symbol] , [symbol] , [symbol] and in FIG. 4 represent:

| | |
|---|---|
| [symbol] G37-113 (3% Tr) | [symbol] G32-104 (3% Tr) - Ref. |
| [symbol] G36-276 (3% Vit E) | [symbol] G36-264 (3% Sol) |

From the values of cumulative percentage of active principle, it is possible to deduce the values of cumulative amount and rate of diffusion.

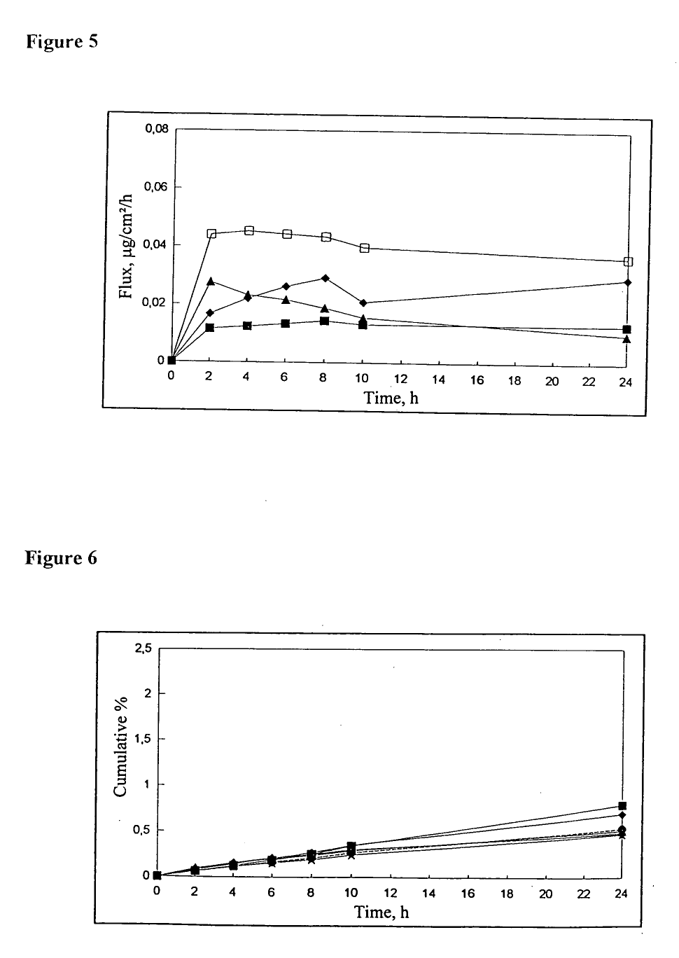

FIG. 5 shows the rate of diffusion of nomegestrol acetate as a function of time.

The symbols ▪, ▴, ♦ and □ in FIG. 5 have the same meaning as in FIG. 3.

The kinetics of diffusion of nomegestrol acetate with the compositions in the form of gels is of the patch type, with constant diffusion.

### Conclusion:

Solketal gives a greater improvement of passage of nomegestrol acetate through the skin than Vitamin E and Transcutol® if we compare the results obtained with those of the G32-104 reference gel.

Thus, better diffusion is obtained using Solketal rather than Vitamin E TPGS, whereas the solubility of the active principle is better for the latter (cf. Tables 5 and 6).

The same comment can be made regarding an aqueous-alcoholic system with the propyleneglycol/Transcutol® mixture: if we consider the four possible combinations 8:0-8:3-3:8 and 0:8, the diffusion obtained is best for the 8:3 mixture. However, solubility is poorest for the latter (cf. Table 7).

The active principle must have some affinity for the solvent if it is to be dissolved completely. However, it must not be too great, so that the partition coefficient between the vehicle and the skin is oriented in favour of diffusion through the skin.

Diffusion tests in static flow with radiolabelled active principle were carried out for gels containing the two types of absorption promoters adopted, versus two reference gels: gel G32-104 (a grade of carbopol different from G37-113 and the other two), for which the best diffusion has been obtained, and gel G37-113, of identical composition to the two gels tested (same grade of Carbopol®) containing Transcutol®.

Examining the action of the absorption promoters adopted, on passage of labelled nomegestrol acetate through the skin, we observe a clear increase in diffusion in the presence of Solketal relative to the G32-104 reference gel containing Transcutol®.

Vitamin E TPGS, used in the same conditions, does not improve passage compared with gel G32-104. On the other hand, if we consider gel G37-113, the diffusion obtained is slightly better with vitamin E, and is increased markedly with Solketal.

When we examine the quantitative distribution of the active principle (cf. FIG. 4) in the structures of the skin, for the active principle of gel G36-264 and of the reference gel G32-104 we find similar concentrations in the epidermis and in the dermis. There is a lower level in the epidermis for gels G37-113 and G36-276.

The above tests also confirm that there is a difference in diffusion of active principle depending on the grade of carbomer used in the formulation (the formulations of gels G37-113 and G32-104 are quantitatively and qualitatively identical apart from this). It appears that diffusion is better in the presence of Carbopol 1382®, just as with distribution in the structures of the skin. Examining the results obtained with respect to adhesion, in tests carried out on these gels G36-264 and G36-276, it is found that the adhesive character of the gel containing Solketal is slightly better than that of the gel containing vitamin E. Note that these two gels contain the same proportions of the excipients, apart from the type of promoter.

### Conclusion

A topical hormonal composition with systemic effect currently preferred according to the present invention is a composition in the form of gel containing:
0.4% nomegestrol acetate
8% propyleneglycol
3% Solketal
0.5% Carbopol 980 or 1382®
45% ethanol 95°
0.05% EDTA, 0.4% TEA and demineralized water to give 100%.

### 2/ Investigations of Formulations in the Form of Film-Forming Solutions

The 5 film-forming solutions investigated were designated << G36-259, G36-261, G36-263, G36-266 and G36-277 >> and their formulations are shown in Table 10 below:

TABLE 10

| | FILM-FORMING SOLUTIONS | | | | |
|---|---|---|---|---|---|
| FORMS | References | | | | |
| Formulations in % | G36-259 | G36-261 | G36-263 | G36-266 | G36-277 |
| Nomegestrol Acetate | 0.4 | 0.4 | 0.4 | 0.4 | 0.4 |
| Propyleneglycol | 8 | 8 | 8 | 8 | 8 |
| Transcutol ® | / | / | / | / | / |
| Solketal | / | / | / | 3 | / |
| Vit E TPGS | / | / | / | / | 3 |
| HPMC 60SH4000 | / | / | / | / | / |
| Carbopol 1382 ® | / | / | / | / | / |
| Carbopol 980 ® | / | / | / | / | / |
| EDTA | 0.05 | 0.05 | 0.05 | 0.05 | 0.05 |
| TEA | / | 0.8 | 0.3 | 0.05 | 0.05 |
| Kollidon 90 ® | 5 | / | / | 5 | 5 |
| Aqoat AS-LF ® | / | 10 | / | / | / |
| Eudragit L30D55 ® | / | / | 10 | / | / |
| Diethyl phthalate | / | 3 | 2 | / | / |
| Ethanol 95 | 43.35 | 40 | 40 | 43.25 | 43.25 |
| Demineralized water | 43.2 | 37.75 | 39.25 | 40.25 | 40.25 |
| pH solution at 1% | 6.25 | 6.16 | 6.24 | 6.83 | 6.34 |
| Viscosity MPa · s | | | | | |
| NAc content (%) | 0.41 | 0.42 | 0.43 | 0.40 | 0.40 |

The main differences in the composition of these film-forming solutions relate to the choice of film-forming agent and the choice of addition, or not, of an absorption promoter or of a plasticizer.

Tests were carried out for the film-forming solutions in comparison with gel G32-104 as reference.

Table 11 shows the cumulative percentage of nomegestrol acetate as a function of time and the distribution of nomegestrol acetate in the cutaneous structures.

TABLE 11

| | Cumulative percentage | | | | | |
|---|---|---|---|---|---|---|
| | GELS | FILM-FORMING SOLUTIONS | | | | |
| | G32-104 | | | | G36-266 | G36-277 |
| | 8% PG | G36-261 | G36-263 | G36-259 | 8% PG | 8% PG |
| Time, | 3% Trans | 8% PG | 8% PG | 8% PG | 3% Solk | 3% TPGS |
| h | 0.5% C1382 | 10% Aqoat | 10% Eudr | 5% Koll | 5% Koll | 5% Koll |
| 0 | 0 | 0 | 0 | 0 | 0 | 0 |
| 2 | 0.062 | 0.088 | 0.092 | 0.077 | 0.067 | 0.064 |
| 4 | 0.123 | 0.153 | 0.152 | 0.14 | 0.116 | 0.112 |
| 6 | 0.185 | 0.206 | 0.197 | 0.203 | 0.165 | 0.153 |
| 8 | 0.252 | 0.251 | 0.239 | 0.269 | 0.211 | 0.193 |
| 10 | 0.342 | 0.3 | 0.289 | 0.349 | 0.269 | 0.242 |
| 24 | 0.799 | 0.487 | 0.515 | 0.699 | 0.539 | 0.474 |
| Epidermis | 8.24 | 5.69 | 2.78 | 9.14 | 6.41 | 4.43 |
| Dermis | 5.56 | 1.93 | 2.26 | 4.58 | 3.2 | 4.76 |
| Washing | 72.18 | 97.96 | 98.85 | 94.33 | 91.37 | 95.24 |

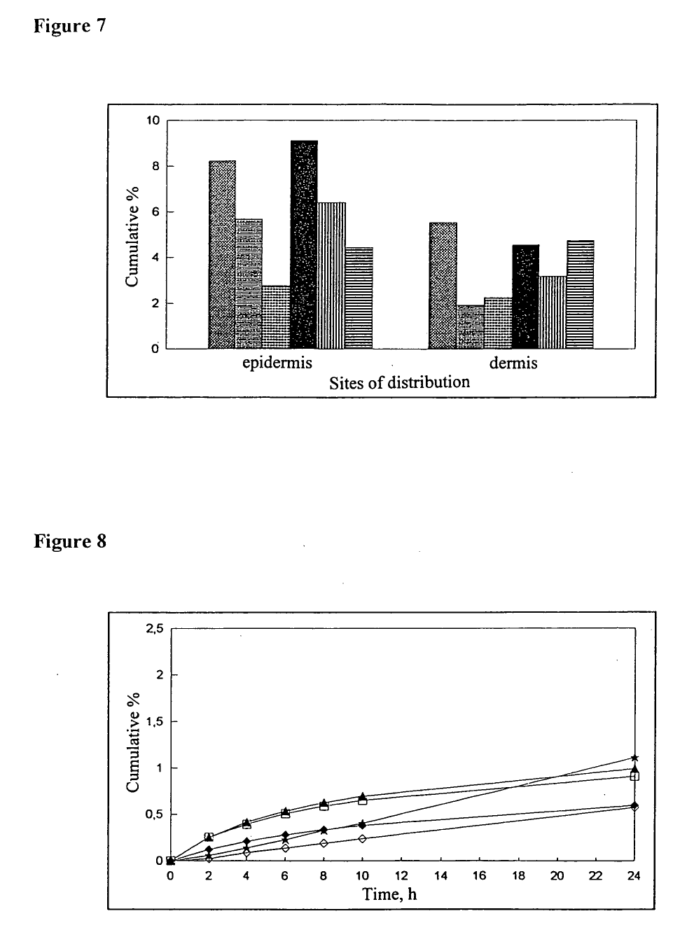

Table 11 is illustrated by FIGS. 6 and 7.

FIG. 6 shows the influence of the absorption promoter and of the film-forming agent on passage of the systemic film of nomegestrol acetate through the skin.

The symbols ▪, ♦, ⋄, , ▴ and ★ in FIG. 6 represent:

| | |
|---|---|
| ▪ G32-104 (3% Tr) -Ref.- | ⋄ G36-261 (10% Aqoat) |
| ♦ G36-259 (5% Kol) | G36-266 (3% Solk/5% Kol) |

▴ G36-263 (10% Eudrag)
★ G36-277 (3% TPGS/5% Kol)

The amounts of active principle that had diffused from these forms are less in all cases than the amounts obtained by application of the non-filming gel G32-104, regardless of the polymer considered.

It can be seen that the solution containing only Kollidon® gives diffusion that is closest to that of the reference gel. The other two polymers produce similar diffusions.

Solutions combining Kollidon® and a promoter, such as Solketal or vitamin E TPGS, give poorer diffusions of active principle compared with solution G36-259 without promoter.

FIG. 7 shows the distribution of nomegestrol acetate in the skin structures.

The symbols [symbol] , , [symbol] , [symbol] , [symbol] and [symbol] in FIG. 7 represent:

| | |
|---|---|
| [symbol] G32-104 (3% Tr) -Ref.- | [symbol] G36-261 (10% Aqoat) |
| [symbol] G36-259 (5% Kol) | [symbol] G36-266 (3% Solk/5% Kol) |

[symbol]

G36-263 (10% Eudrag) [symbol]
G36-277 (3% TPGS/5% Kol)

It can be seen that distribution is best for Kollidon®. It is equivalent to that found for the reference gel. The results obtained for the solutions of Aqoat® and of Eudragit® remain low.

The diffusion results obtained with the film-forming solutions are slightly better than those obtained with the gel with 0.11% of nomegestrol acetate (cf. Table 1, example I). However, it should be noted that with regard to Eudragit® and Aqoat®, the solutions prepared only contain propyleneglycol, without any other promoter, in contrast to the reference gel.

### Conclusion:

A topical hormonal composition with systemic effect according to the present invention will be, for example, a composition in the form of film-forming solution containing:
0.4% of nomegestrol acetate
8% of propyleneglycol
5% of Kollidon 90®
43.35% of 95° ethanol
0.05% of EDTA and demineralized water to give 100%.

### 3/ Investigation of Formulations in the Form of Film-Forming Gels or Gelled Films

The three film-forming gels investigated were designated << G36-260, G36-262 and G36-267 >> and their formulations are shown in Table 12 below.

TABLE 12

| | Film-forming GELS | | |
|---|---|---|---|
| FORMS | References | | |
| Formulations in % | G36-260 | G36-262 | G36-267 |
| Nomegestrol Acetate | 0.4 | 0.4 | 0.4 |
| Propyleneglycol | 8 | 8 | 8 |
| Transcutol ® | / | / | / |
| Solketal | / | / | / |
| Vit E TPGS | / | / | / |
| HPMC 60SH4000 | / | / | 1 |
| Carbopol 1382 ® | / | / | / |
| Carbopol 980 ® | 0.5 | 0.75 | / |
| EDTA | 0.05 | 0.05 | 0.05 |
| TEA | 0.1 | 0.9 | 0.4 |
| Kollidon 90 ® | 5 | / | / |
| Aqoat AS-LF ® | / | 10 | / |
| Eudragit L30D55 ® | / | / | 10 |
| Diethyl phthalate | / | 3 | 2 |
| Ethanol 95 | 43 | 40 | 40 |
| Demineralized water | 42.95 | 36.9 | 38.15 |
| pH solution at 1% | 6.36 | 6.2 | 6.17 |
| Viscosity mPa · s | 1750 | 1050 | 1150 |
| NAc content (%) | 0.41 | 0.405 | 0.40 |

The main differences in the composition of these film-forming gels relate to the choice of gelling agent and of film-forming agent.

These tests were carried out for the film-forming gels in comparison with gels G32-104 and G37-113 as reference.

Table 13 shows the cumulative percentage of nomegestrol acetate as a function of time and the distribution of nomegestrol acetate in the structures of the skin.

| | % Cumulative | | | | |
|---|---|---|---|---|---|
| | GEL | | FILM-FORMING GEL | | |
| | G32-104 | G37-113 | G36-260 | G36-262 | G36-267 |
| | 8% PG | 8% PG | 8% PG | 8% PG | 8% PG |
| Time | 3% Trans | 3% Trans | 5% Kollidon | 10% aqoat | 10% Eudr |
| h | 0.5% C1382 | 0.6% C980 | 0.5% C980 | 0.75% C980 | 1% HPMC |
| 0 | 0.000 | 0.000 | 0.000 | 0.000 | 0.000 |
| 2 | 0.058 | 0.024 | 0.121 | 0.251 | 0.251 |
| 4 | 0.135 | 0.088 | 0.206 | 0.414 | 0.392 |
| 6 | 0.227 | 0.137 | 0.278 | 0.533 | 0.506 |
| 8 | 0.329 | 0.190 | 0.334 | 0.626 | 0.590 |
| 10 | 0.402 | 0.239 | 0.379 | 0.694 | 0.650 |
| 24 | 1.117 | 0.575 | 0.598 | 0.994 | 0.913 |
| Epidermis | 14.82 | 10.05 | 11.42 | 16.36 | 11.21 |
| Dermis | 6.97 | 4.33 | 4.81 | 1.53 | 1.39 |
| Washing | 61.15 | 67.63 | 65.83 | 72.35 | 90.42 |

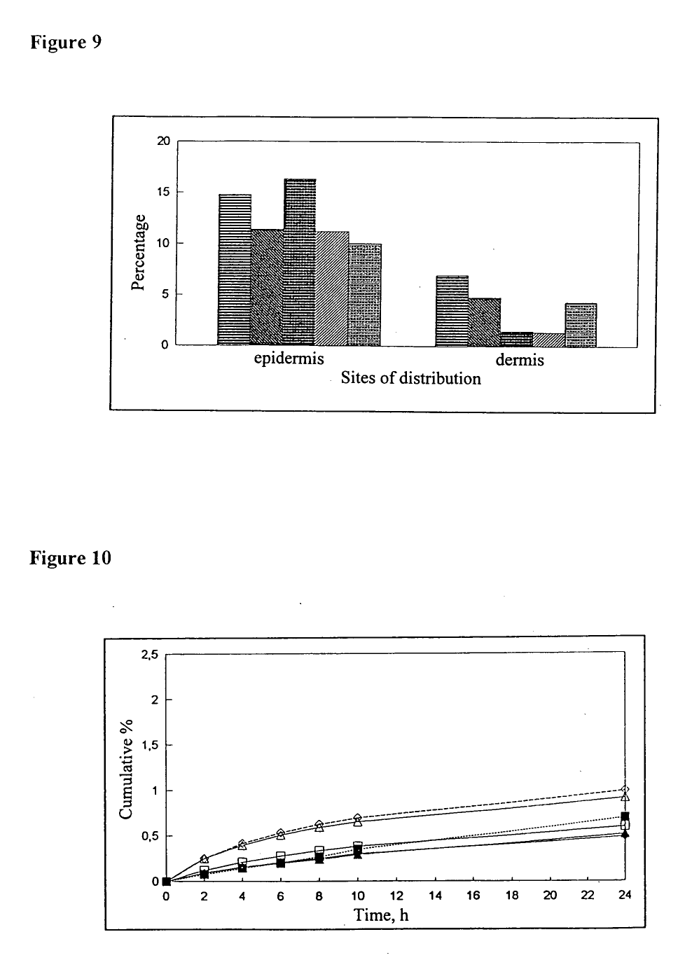

Table 13 is illustrated by FIGS. 8 and 9.

FIG. 8 shows the influence of the film-forming agent on passage of the systemic film-forming gel of nomegestrol acetate through the skin.

The symbols ★, □, ♦, ⋄ and ▴ in FIG. 8 represent:

| | | |
|---|---|---|
| ★ G32-104 (C1382) | ♦ G36-260 (Kol/C980) | ▴ G36-262 |
| □ G36-267 (Eud/HPMC) | ⋄ G37-113 (C980) | (aq/C980) |

Examining all of the polymers, the diffusion of nomegestrol acetate from film-forming gels of Aqoat® and of Eudragit® is better than for the G32-104 ((reference)) gel up to 10 hours. Beyond that, the trend is slightly reversed. If we consider the non-filming gel 113, containing a different carbopol® from that in gel G32-104, the results obtained for all the film-forming gels are better, regardless of the polymer considered.

Note that diffusion of the active principle is similar for Aqoat® and Eudragit®.

On the other hand, it is much lower for Kollidon®.

FIG. 9 shows the distribution of the active principle in the structures of the skin.

The symbols [symbol] , [symbol] , [symbol] , [symbol] , [symbol] in FIG. 9 represent:

| | | |
|---|---|---|
| [symbol] G32-104 (C1382) | [symbol] G36-260 (Kol/C980) | [symbol] G36-262 |
| [symbol] G36-267 (Eud/HPMC) | [symbol] G37-113 (C980) | (aq/C980) |

It can be seen that the distribution varies from one polymer to another: relative to the G32-104 reference gel, the distribution in the epidermis is similar for Aqoat®, but lower for Kollidon® and Eudragit®. The distribution in the dermis is lower for Aqoat® and Eudragit®, but higher for Kollidon®.

### Conclusion:

Examples of topical hormonal compositions with systemic effect according to the invention are, for example, compositions in the form of film-forming gel containing:
0.4% of nomegestrol acetate
8% of propyleneglycol
0.75% of Carbopol 980®
10% of Aqoat AS-LF®
40% of ethanol 95°
3% of diethyl phthalate, 0.05% of EDTA, 0.9% of TEA and demineralized water to 100%,
   or, compositions in the form of film-forming gel containing:
0.4% of nomegestrol acetate
8% of propyleneglycol
1% of HPMC 60 SH 4000
10% of Eudragit L 30 D 55®
40% of ethanol 95°
2% of diethyl phthalate, 0.05% of EDTA, 0.4% of TEA and demineralized water to 100%.

### 4/ Comparison Between Film-Forming Solutions and Film-Forming Gels

Table 14 shows the cumulative percentage of nomegestrol acetate as a function of time, and the distribution of nomegestrol acetate in the structures of the skin.

| | % cumulative | | | | | |
|---|---|---|---|---|---|---|
| | | | | FILM-FORMING GELS | | |
| | FILM-FORMING SOLUTIONS | | | G36-260 | G36-262 | G36-267 |
| | G36-259 | G36-261 | G36-263 | 8% PG | 8% PG | 8% PG |
| Time | 8% PG | 8% PG | 8% PG | 5% Koll | 10% Aqoat | 10% Eudr |
| h | 5% Koll | 10% Aqoat | 10% Eudr | 0.5% C980 | 0.75% C980 | 1% HPMC |
| 0 | 0 | 0 | 0 | 0 | 0 | 0 |
| 2 | 0.077 | 0.088 | 0.092 | 0.121 | 0.251 | 0.251 |
| 4 | 0.14 | 0.153 | 0.152 | 0.206 | 0.414 | 0.392 |
| 6 | 0.203 | 0.206 | 0.197 | 0.276 | 0.533 | 0.506 |
| 8 | 0.269 | 0.251 | 0.239 | 0.334 | 0.626 | 0.59 |
| 10 | 0.349 | 0.3 | 0.289 | 0.379 | 0.694 | 0.65 |
| 24 | 0.699 | 0.487 | 0.515 | 0.598 | 0.994 | 0.913 |
| Epidermis | 9.14 | 5.69 | 2.78 | 11.42 | 16.36 | 11.21 |
| Dermis | 4.58 | 1.93 | 2.26 | 4.81 | 1.53 | 1.39 |
| Washing | 94.33 | 97.96 | 98.85 | 65.83 | 72.35 | 90.42 |

Table 14 is illustrated by FIGS. 10 and 11.

FIG. 10 makes it possible to compare film-forming solutions and film-forming gels of nomegestrol acetate with systemic effects.

The symbols ▪, □, ♦, ⋄, ▴ and Δ in FIG. 10 represent:

Film-forming solutions

| | | |
|---|---|---|
| ▪ G36-259 | ♦ G36-261 | ▴ G36-263 |
| (5% Kollidon) | (10% Aqoat) | (10% Eudrag) |

Film-forming gels

| | | |
|---|---|---|
| □ G36-260 | ⋄ G36-262 | Δ G36-267 |
| (5% Kol/C980) | (10% Aqoat/C980) | (10% Eudr/HPMC) |

FIG. 11 shows the distribution of the active principle in the structures of the skin.

The symbols , [symbol] , [symbol] (column 2, FIG. 11), [symbol] [symbol] (column 3, FIG. 11) and [symbol] (last column) in FIG. 11 represent

| | | |
|---|---|---|
| [symbol] G36-259 | [symbol] G36-261 | [symbol] G36-263 |
| (5% Kollidon) | (10% Aqoat) | (10% Eudrag) |
| [symbol] G36-260 | [symbol] G36-262 | [symbol] G36-267 |
| (5% Kol/C980) | (10% Aqoat/C980) | (10% Eudr/HPMC) |

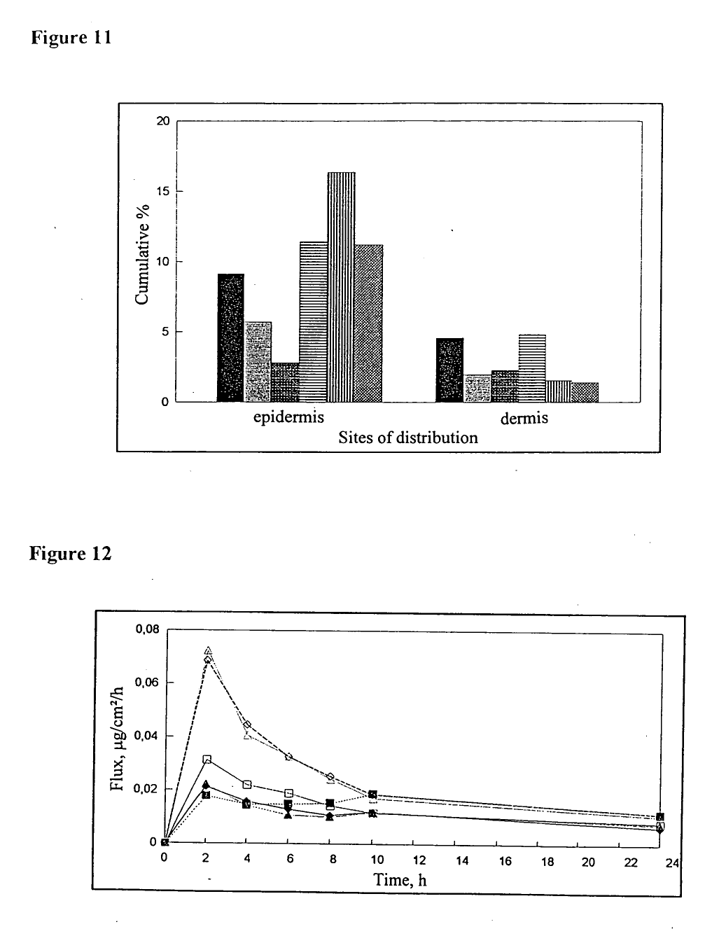

FIG. 12 compares the diffusion of nomegestrol acetate with compositions in the form of film-forming gel and with compositions in the form of film-forming solution.

The symbols in FIG. 12 have the same meanings as in FIG. 10.

In contrast to FIG. 5 (diffusion flux with compositions in the form of gel), the kinetics of diffusion is not kinetics with constant diffusion, but very quickly (after 2 hours) displays maximum diffusion which then decreases quite quickly. This is especially true of the film-forming gels G36-262 and G36-267. Thus, two types of flux can be distinguished: flux with more or less constant diffusion, and other forms which very quickly exhibit a maximum diffusion peak.

Thus, the film-forming gels are better suited than a film-forming solution for optimization of percutaneous distribution of nomegestrol acetate. More particularly, the mere presence of a cellulosic (Aqoat®, G36-262) or acrylic (Eudragit®, G36-267) film-forming agent in a film-forming gel makes it possible to achieve good diffusion of the active principle. The film that forms, in both cases, is stronger, more cohesive, and seems to permit release of the active principle.

Accordingly, it is possible that topical hormonal compositions with systemic effect in the form of film-forming gel can be combined with 3% of Solketal to obtain a synergistic action and further improve the diffusion of nomegestrol acetate.

### 4/ Conclusion

Film-forming or filming solutions generally only permit diffusion of active principle less than that obtained for the reference gel (G32-104).

On the other hand, film-forming gels of Aqoat® (G36-262) and of Eudragit® (G36-267) can provide considerable diffusion of active principle, if we consider the formulations that do not contain any absorption promoter.

Solketal is an absorption promoter that seems to have an action on the diffusion of nomegestrol acetate through the skin; thus, in an aqueous-alcoholic system and when combined with propyleneglycol in the proportions (3:8), it greatly improves its solubility in the vehicle and its passage through the skin.

Thus, a particularly suitable example of a topical hormonal composition with systemic effect according to the invention is a composition that is in the form of a gel or a film-forming gel and contains, in an aqueous-alcoholic mixture, 8% of propyleneglycol and 3% of isopropylidene glycerol.

### Example IV

### Preliminary Clinical Trials

In these examples, clinical trials were carried out on women, with the gel containing 0.11% of nomegestrol acetate, whose formulation is given in Table 1 of example I.

### 1/ Clinical Example No. 1

Twenty-four volunteers, women in good health and in the period of ovarian activity, with average age of 23.5 years, were treated on 15 consecutive days with 4 mg of nomegestrol acetate in a gel applied every day to both breasts.

Repeated blood samples were taken in the hours following the first and last administration, on 9 occasions (6 times before application of the gel and 3 times 3 hours afterwards), between the 2^{nd} and the 14th day of treatment. The plasma of these samples was analysed for nomegestrol acetate by liquid chromatography combined with mass spectrometry.

From the very first day of treatment, nomegestrol acetate was detected in all the subjects. The maximum concentration was evaluated at 0.25±0.027 ng/ml and the area under the curve from 0 to 48 hours, of 6.08±0.775 ng/ml per h, the mean values forming a plateau between 0.10 and 0.17 ng/ml.

After the last administration, the maximum concentration was 0.65±0.073 ng/ml and the area under the curve from 0 to 48 hours was 18.43±2.091 ng/ml per h, nomegestrol acetate still being detected in the plasma 72 hours after the last application (at a level of 0.19±0.027 ng/ml).

A state of equilibrium is obtained after the 3^{rd} day of treatment. We then observe mean values that remain on a plateau, with little variation, between 0.42 and 0.65 ng/ml. pos 2/ Clinical Example No. 2

Six menopausal women, with ages ranging from 56 to 66 years and without hormone replacement treatment for 2 months, were monitored for 2 consecutive cycles of 25 days, separated by a therapeutic window of 6 days.

During each day, they were given one tablet of oral oestradiol per day and, during the 15 days of the second cycle, 4 mg of nomegestrol acetate applied as a gel on the abdominal skin. At the end of each cycle, the plasma was analysed for nomegestrol acetate, the occurrence of genital bleeding was noted and a biopsy of the endometrium was carried out.

In contrast to what was observed in the first cycle (oestradiol alone), during the second cycle, administration of the gel of nomegestrol acetate showed that the progestogen could be detected in the plasma at levels between 0.39 and 0.76 ng/ml (0.62 ng/ml on average) and that these levels were sufficient to cause secretory transformation of the endometrium and produce genital bleeding, on average 5 days after the end of the second cycle.

### 3/ Clinical Example No. 3

One hundred and thirteen premenopausal women, who had been suffering from breast pains for more than 3 months and for at least 7 days per cycle, were treated for an average time of 130 days with 4 mg of nomegestrol acetate applied each day, on the last 15 days of the menstrual cycle, in the form of a gel on both breasts.

Efficacy was assessed after 3 months and at the end of treatment using a visual analogue scale for quantifying breast pain.

This evaluation demonstrated that the nomegestrol acetate gel led to a statistically significant decrease in the intensity and duration of the cycle of breast pain, starting from the 3^{rd} month of treatment. After 6 cycles of treatment, the intensity had decreased by 48% and the duration by 41%.

In the course of this study, 55 women were analysed for nomegestrol acetate in the blood, and values of 0.44±0.30 (m±sd) ng/ml were obtained.

1-17. (canceled)

18. A method for insuring a hormonal substitution therapy in menopaused women subjected to an estrogenotherapy for substitution, comprising applying on the skin of said women a safe but effective amount of a gel or film-forming gel, endowed with systemic activity, based on Nomegestrol, its esters or its ethers.

19. A method of claim 18 for restauring genital bleeding in menopausal women comprising administering on the skin of said women a safe but efficient amount of a gel based on Nomegestrol, its ethers or its esters, sufficient to cause secretory transformation of the endometrium and to produce genital bleedings.

20. A method of claim 18 wherein the gel or film-forming gel comprising
a) Nomegestrol acetate,
b) a gel-forming agent selected from the group consisting of hydroxypropyl methyl cellulose and a carbomer,
c) a solubilising non volatile agent selected from the group consisting of alkanols, polyethylene glycol, propylene glycol, polyoxethylated glycosyl glycerids, Labrasol® and water,
d) an absorption promoter selected from the group consisting of isopropylideneglycerol and di-ethylene glycol mono-ethyl ether, and
e) a film-forming agent selected from the group consisting of cellulose derivatives, methacrylic derivatives, and polyvinyl pyrrolidone derivatives.

21. A method of claim 18 wherein the gel or film-forming gel comprising
a) Nomegestrol acetate,
b) a gel-forming agent selected from the group consisting of hydroxypropyl methyl cellulose and a carbomer,
c) a solubilising non volatile agent selected from the group consisting of alkanols, polyethylene glycol, propylene glycol, polyoxethylated glycosyl glycerids, Labrasol® and water,
d) an absorption promoter selected from the group consisting of isopropylideneglycerol and di-ethylene glycol mono-ethyl ether, and
e) a film-forming agent selected from the group consisting of cellulose derivatives, methacrylic derivatives, and polyvinyl pyrrolidone derivatives.

22. A method of claim 18 wherein the gel or film forming composition is based on
a) Nomegestrol Acetate at a concentration of 0.1 to 0.8%,
b) Propylene glycol at a concentration of 2 to 20%,
c) Isopropylidene glycerol at a concentration of 3%,
d) a carbomer selected from the group consisting of Carbopol® 980 and Carbopol®
e) a complexing agent
f) 95° ethanol and
g) Water.

23. A method of claim 18 wherein the gel or film forming gel is based on
a) Nomegestrol acetate at a concentration of 0.1 to 0.8%,
b) Propyleneglycol,
c) a carbomer
d) a film forming agent of cellulosic nature sold under the trade name aquoat at AS-LF
e) diethyl phtalate
f) ethylene diaminotetraacetic acid
g) triethanol amine (TEA)
h) isopropylidene glycerol
i) Ethanol
j) Water

24. A method of claim 18 wherein the gel or film forming gel is based on
a) Nomegestrol acetate at a concentration of 0.1 to 0.8%,
b) Propylene glycol,
c) Polyvinyl pyrrolidone,
d) 95° ethanol,
e) Water and
f) Ethylene diaminotetracetic acid.
